(19) United States
(12) Patent Application Publication
(10) Pub. No.: US 2026/0080997 A1
(43) Pub. Date: Mar. 19, 2026

(54) BLOCKCHAIN BASED SYSTEM AND METHOD FOR REGULATION AND INCENTIVIZING MEDICATION MANAGEMENT

(71) Applicants: Sambhu N. Choudhury, Cincinnati, OH (US); Lewis Charles Smyrnios, Satellite Beach, FL (US); Shayon Choudhury, Cincinnati, OH (US)

(72) Inventors: Sambhu N. Choudhury, Cincinnati, OH (US); Lewis Charles Smyrnios, Satellite Beach, FL (US); Shayon Choudhury, Cincinnati, OH (US)

(21) Appl. No.: 19/331,647

(22) Filed: Sep. 17, 2025

Related U.S. Application Data
(60) Provisional application No. 63/696,018, filed on Sep. 18, 2024.

(51) Int. Cl. G16H 20/13; G06F 21/32; G07C 9/00; G16H 10/60

(52) U.S. Cl. CPC G16H 20/13; G06F 21/32; G07C 9/00571; G16H 10/60

(57) ABSTRACT

A system and methods for medication management and incentivized compliance using a distributed ledger are disclosed. A medication dispenser with a lock, weight sensor, and control unit communicates with a blockchain network executing smart contracts that authorize dispensing events, record authenticated telemetry, and account for tokenized dose entitlements normalized in morphine-milligram equivalents (MME). A prescriber device assigns entitlements to a patient wallet; the dispenser unlocks only upon receipt of an on-chain authorization event that cryptographically binds the requested dose to measured inventory and policy constraints. Post-dispense, signed weight deltas are committed to an authenticated store for audit. Optional risk scoring derived from historical usage features gates dispensing or escalates to multi-party authorization. Unused entitlements may be reconciled under policy via redemption logic. The architecture reduces diversion by coupling cryptographic authorization with tamper-evident telemetry and immutable audit trails.

### CROSS-REFERENCE TO RELATED APPLICATIONS

The present Application claims priority to U.S. Provisional Application Ser. No. 63/696,018 entitled “BLOCKCHAIN BASED SYSTEM AND METHOD FOR REGULATION AND INCENTIVIZING MEDICATION MANAGEMENT”, filed Sep. 18, 2024, which is hereby expressly incorporated by reference herein for all purposes.

### TECHNICAL FIELD

The present disclosure relates generally to medication management, and more particularly, to a blockchain based system and method for medication management and incentivizing medication management and regulation.

### BACKGROUND

### Medication Regulation

Opioid drugs are known for their powerful analgesic properties and are primarily used to alleviate pain. The effect of administration of opioids further includes drowsiness, changes in mood and alterations of the endocrine and autonomic nervous systems. The drug alters the mood in a manner to provide a desirable sense of “well-being” dissociated from therapeutic ameliorative effects. This mood-altering effect is extremely pleasurable for some individuals, which leads to development of craving for re-administration of the opioid and results in opioid addiction.

Opioid addicts can obtain drugs from a variety of illicit sources. The street drugs are of questionable quality. Therefore, to potential abusers, prescription pharmaceutical opioids can be particularly attractive as a drug source because of their high purity and dependable dosage.

This is especially true in the case of extended-release opioid dosage forms. Extended-release opioid dosage forms are intended for decreased, frequency of dosing. Therefore, each tablet must contain the number of opioids, which would be contained in several immediate release tablets. Narcotic pills are also not all the same with different medications and brands having additional ingredients including acetaminophen for potentiation. In other cases, one pill is not the same as the other such as Vicodin®, which contains the opioid hydrocodone, and Percocet®, which contains the opioid oxycodone. These drugs have differing amounts of narcotics so they should be considered as narcotic equivalents rather than managed as individual pills.

This results in the production of dosage forms having substantially increased amounts of opioid. A single extended-release tablet can provide much more opioid to the potential abuser than low dose, immediate release dosage forms. This results in stronger feeling of euphoria, or “high” from controlled release tablets than an abuser would get from an immediate release tablet. This makes such tablets more desirable for an abuser. Further, in some people, opioids cause addiction, even when the medications are prescribed appropriately and taken as directed.

Numerous forms of opioid drugs are developed to prevent addiction. However, self-control is an important factor to resist temptation and to prevent relapse of individuals recovered from drug addiction.

### Distributed Ledger Technology (DLT)

A distributed ledger (also called a shared ledger or distributed ledger technology or “DLT”) is a digital system for recording the transaction of assets in which the transactions and their details are recorded in multiple places at the same time. Unlike traditional databases, distributed ledgers have no central data store or administration functionality.

DLT refers specifically to the technological infrastructure and protocols that enable the simultaneous access, validation and updating of records that characterize distributed ledgers. It works on a computer network spread over multiple entities, locations or nodes.

As used herein, “distributed ledger technologies” (DLTs) include blockchains, directed acyclic graphs (DAGs), hashgraphs, and other peer-to-peer ledger architectures that maintain a tamper-evident, append-only record across a plurality of networked nodes. In certain implementations, each node maintains a copy of the ledger and processes and verifies each submitted item (e.g., transaction, event, or state update) according to predetermined validation rules, thereby generating a cryptographic record of the item and achieving consensus on its veracity via a consensus protocol (e.g., proof-of-work, proof-of-stake, practical Byzantine fault tolerance, or gossip-based virtual voting). A DLT can be used to record static data, such as registries, attestations, or configuration states, as well as dynamic data, such as financial transfers, asset movements, or smart-contract state transitions. Cryptographic primitives, including collision-resistant hash functions, digital signatures, and, in some embodiments, Merkle-style commitment structures, provide integrity, authenticity, and efficient auditability. Blockchain is a well-known example of a DLT, typically employing a secure hash algorithm (SHA, e.g., SHA-256 or SHA-3) to link blocks of validated items in chronological order; analogous assurances of integrity and consensus are provided in DAG-, hashgraph-, and related ledger topologies, whether deployed in permissionless or permissioned networks.

Ethereum uses Keccak-256 (pre-standardized Keccak), which takes an input of an arbitrary length and produces a fixed-length output of 256 bits. A directed acyclic graph is a graph that has vertices and edges with each edge directed from one vertex to another and not directed cycles. A directed acyclic graph is topologically ordered by arranging the vertices as a linear ordering, which can provide chronological transaction record ordering where the transactions can be used to track payments, accounts, smart contracts, etc. A hashgraph includes a system of individual nodes in a network that share transaction messages to create directed acyclic graphs that time-sequence transactions where each message contains one or more transactions, a timestamp, a digital signature and cryptographic hashes of earlier events.

DLT works based on principles of decentralization. Unlike traditional centralized databases, DLT operates on a peer-to-peer (P2P) network, where multiple nodes store, validate and update the ledger simultaneously. This eliminates the need for a central authority and reduces the risk of a single point of failure. The process begins with the replication of digital data across the network of nodes. Each node maintains an identical copy of the ledger and independently processes new update transactions. To ensure consensus, all participating nodes employ a consensus algorithm that determines the correct version of the ledger. Once a consensus is reached, the updated ledger is propagated to all nodes, ensuring synchronization and accuracy.

DLT uses cryptography to securely store data and cryptographic signatures and keys to allow access only to authorized users. The technology also creates an immutable database, which means information, once stored, cannot be deleted and any updates are permanently recorded for posterity.

This architecture represents a significant change in how information is gathered and communicated by moving record-keeping from a single, authoritative location to a decentralized system in which all relevant entities can view and modify the ledger. As a result, all other entities can see who is using and modifying the ledger. This transparency of DLT provides a high level of trust among the participants and practically eliminates the chance of fraudulent activities occurring in the ledger.

As such, DLT removes the need for entities using the ledger to rely on a trusted central authority that controls the ledger or an outside, third-party provider to perform that role and act as a check against manipulation.

### Tokens

Generally speaking, a token is a representation of a particular asset or utility. Within the context of blockchain technology, tokenization is the process of converting something of value into a digital token that's usable on a blockchain application. Assets tokenized on the blockchain come in two forms. They can represent tangible assets like gold, real estate, and art, or intangible assets like voting rights, ownership rights, or content licensing. Practically anything can be tokenized if it is considered an asset that can be owned and has value to someone, and can be incorporated into a larger asset market.

In addition to the above classifications, tokens can also be designed to be either fungible or non-fungible, depending on their intended use. Fungible tokens are identical and can seamlessly replace one another. On the other hand, non-fungible tokens (NFTs) are unique and provably scarce, meaning their histories can be traced down to the individual level.

Conventional medication access systems often rely on server-side polling and post-hoc review, which provide limited guarantees at the point of dispensing. Authorization is not cryptographically bound to the measured device state, making replay and stale approvals difficult to prevent. Offline operation can lead to inconsistent records without deterministic reconciliation, and audit trails may be incomplete or mutable. These limitations make it challenging to enforce policy windows, detect tampering in real time, and furnish verifiable evidence of correct operation.

There is a currently a need for a system and method for medication management and incentivizing medication management using traceable technology. Further, there is a need of a system and method that induces the users of opioid to be self-conscious about the usage of medication.

The disclosed architecture addresses these technical limitations by cryptographically binding authorization to device-attested state, enforcing windowed policy at the edge, and committing Merkle-authenticated telemetry to produce a tamper-evident audit trail.

### SUMMARY OF THE INVENTION

The disclosed subject matter relates to ledger data structures. The disclosed subject matter also relates to tracking digital data associated with medication management and incentivizing medication management. The system comprises a medication dispenser comprising at least one compartment, a control unit, a weighing unit and a dispensing unit. The compartment is configured to store a quantity of medication. The medication dispenser is associated with a first user. The system further comprises a blockchain network in communication with the dispenser. The system further comprises a first user device in communication with the blockchain network and the dispenser. The first user device is associated with the first user. The system further comprises a second user device in communication with the blockchain network. The second user device is associated with a second user. In one embodiment, the first user includes a patient and the second user includes a healthcare provider. The blockchain network comprises a plurality of decentralized computing nodes. In one embodiment, the distributed ledger is blockchain network comprising a blockchain network capable of supporting and executing automated, self-regulating contracts between two or more parties (“smart contracts”).

In the blockchain ecosystem, tokens are assets that allow information and value to be transferred, stored, and verified in an efficient and secure manner. Crypto tokens can take many forms and can be programmed with unique characteristics that expand their use cases.

In general, a token is a representation of a particular asset or utility. Within the context of blockchain technology, tokenization is the process of converting something of value into a digital token that's usable on a blockchain application. Assets tokenized on the blockchain come in two forms. They can represent tangible assets like gold, real estate, and art, or intangible assets like voting rights, ownership rights, or content licensing.

Crypto tokens enable both information and value to be transferred, stored, and verified in a way that is both efficient and secure. Tokens can also be designed to be either fungible or non-fungible, depending on their intended use. Fungible tokens are identical and can seamlessly replace one another. On the other hand, non-fungible tokens (NFTs) are unique and provably scarce, meaning their histories can be traced down to the individual level.

The blockchain network is configured to enable the second user to prescribe a quantity of medication to be filled in the compartment and transfer a number of tokens corresponding to the quantity of medication prescribed to the first user, via the second user device. Each token comprises a monetary value. The blockchain network is configured to receive a request from the first user, via the first user device, to dispense a quantity of medication. The blockchain network is configured to retrieve at least one of an information corresponding to a quantity of medication stored after a previous dispense period and information corresponding to the quantity of medication present in the compartment. The blockchain network is further configured to retrieve the information related to the requested quantity of medication for a current period of time. The blockchain network is further configured to determine if the requested quantity of medication is within a predefined limit for the current period of time. The blockchain network is further configured to approve the request to dispense the requested quantity of medication when the requested quantity of medication is within the predefined limit for the current period of time.

The blockchain network is further configured to record information related to the quantity of medication present in the compartment after each dispensing period and information related to the request from the first user to dispense the quantity of medication in a distributed ledger. The blockchain network is further configured to update the tokens assigned to the first user based on the dispensed quantity of medication. The blockchain network is further configured to enable the first user to trade the tokens corresponding to unused quantity of medication for a monetary value with a guidance of the second user. The blockchain network is further configured to track and report each action of the first user. The blockchain network is further configured to track and report date and timestamp of the functions.

In one embodiment, the medication comprises opioid drugs and the quantity of medication is a measure of morphine-equivalent units of the opioid drugs. The medication dispenser further comprises a locking unit configured to lock the dispensing unit to prevent dispensing of medications without authorization. The control unit is configured to enable the locking unit to unlock the dispensing unit based on the approval of the request received from the blockchain network. The control unit is further configured to enable the dispensing unit to dispense the requested quantity of medication. The weighing unit comprises a weight sensor configured to transmit information related to the quantity of medication in the compartment.

In one embodiment, the medication dispenser is linked to the first user device via an authentication system. The authentication system includes at least one of a biometric scan-based authentication system, a two-factor, or a multi-factor authentication system. The authentication system also includes any other additional electronic authentication or third party authentication. The system further comprises a third user device in communication with the blockchain network. The third user device is associated with a third user. The blockchain network is configured to enable the third user to allocate and transfer a predefined number of tokens for a predefined quantity of medication to the second user. The third user is an administrator. The blockchain network is further configured to create and store a usage history for each patient at the blockchain, and send alert regarding the usage of medication to at least one of the first user, the second user, the third user and a caretaker. The blockchain network 106 includes at least one of public, a permissioned, and a hybrid (public/private) blockchain. The system further comprises an artificial intelligence module configured to analyze and process patient data, including patient history, prior usage, socioeconomic data, case example data, and police data. The artificial intelligence module utilizes standards of usage and addiction to guide subsequent prescription decisions. The blockchain network is further configured to store, report, and update patient data for each interaction. Each interaction contributes to the enhancement of the system's intelligence.

The present invention discloses a blockchain based method for medication management and incentivizing medication management. The method of the present invention is executed in a system comprising a medication dispenser comprising at least one compartment, a control unit, a weighing unit and a dispensing unit. The compartment is configured to store a quantity of medication. The medication dispenser is associated with a first user. The system further comprises a blockchain network in communication with the dispenser. The system further comprises a first user device in communication with the blockchain network and the dispenser. The first user device is associated with the first user. The system further comprises a second user device in communication with the blockchain network. The second user device is associated with a second user. In one embodiment, the first user includes the patient and the second user includes the healthcare provider. The system further comprises a third user device in communication with the blockchain network. The third user device is associated with a third user. The third user is an administrator. In one embodiment, the administrator could be a pharmacy, drug manufacturer or any other entity.

In one embodiment, the medication dispenser is linked to the first user device via an authentication system. The authentication system includes at least one of a biometric scan-based authentication system or a two-factor authentication system. At one step, the blockchain network is configured to enable the second user to prescribe a quantity of medication to be filled in the compartment and transfer a number of tokens corresponding to the quantity of medication prescribed to the first user, via the second user device. Unused entitlements are reconciled under policy via burn, prescriber-directed reissue to a replacement package, or redemption into a non-monetary compliance credit or care-coordination benefit that is recorded on-chain and is non-transferable and not convertible to currency. At another step, the blockchain network is configured to receive a request from the first user, via the first user device, to dispense a quantity of medication.

At yet another step, the blockchain network is configured to retrieve at least one of an information corresponding to a quantity of medication stored after a previous dispense period and information corresponding to the quantity of medication present in the compartment. At yet another step, the blockchain network is configured to retrieve the information related to the requested quantity of medication for a current period of time. At yet another step, the blockchain network is configured to determine if the requested quantity of medication is within a predefined limit for the current period of time. At yet another step, the blockchain network is configured to approve the request to dispense the requested quantity of medication when the requested quantity of medication is within the predefined limit for the current period of time.

At yet another step, the blockchain network is configured to record information related to the quantity of medication present in the compartment after each dispensing period and information related to the request from the first user to dispense the quantity of medication in a ledger. At yet another step, the blockchain network is configured to update the tokens assigned to the first user based on the dispensed quantity of medication. At yet another step, the blockchain network is configured to enable the first user to trade the tokens corresponding to unused quantity of medication for a monetary value with a guidance of the second user. In one embodiment, the medication comprises opioid drugs and the quantity of medication is a measure of morphine-equivalent units of the opioid drugs. The medication dispenser further comprises a locking unit. At yet another step, the locking unit is configured to lock the dispensing unit to prevent dispense of medications without authorization.

At yet another step, the control unit is configured to enable the locking unit to unlock the dispensing unit based on the approval of the request received from the blockchain network. At yet another step, the control unit is configured to enable the dispensing unit to dispense the requested quantity of medication. At yet another step, the weighing unit is configured to transmit information related to the quantity of medication in the compartment. The weighing unit comprises a weight sensor. At yet another step, the blockchain network is configured to enable the third user to allocate and transfer a predefined number of tokens for a predefined quantity of medication to the second user. At yet another step, the blockchain network is configured to create and store a usage history for each patient at the distributed ledger/blockchain protocol, and send alert regarding the usage of medication to at least one of the first user, the second user, the third user and a caretaker. The blockchain network is configured to issue alerts or reports in the event of deviations in dispenser weight from the expected value. The deviation of dispenser weight could be determined using the weight sensor and usage history of medication. The blockchain network is further configured to generates alerts or reports for unauthorized access or tampering. The blockchain network is further configured to store and update patient data for each interaction. Each interaction contributes to the enhancement of the system's intelligence. At yet another step, an artificial intelligence module is configured to analyze and process patient data, including patient history, prior usage, socioeconomic data, case example data, and police data. The artificial intelligence module utilizes standards of usage and addiction to guide subsequent prescription decisions. Current artificial intelligence (AI) technologies function within a set of pre-determined parameters. Artificial general intelligence (AGI) may also be used, which is a field of AI that attempts to create software with human-like intelligence and the ability to self-teach.

In one embodiment, the system allows the management of prescribers of medications as individuals such that the blockchain will track and manage the prescribing practices of individual prescribers

In one embodiment, the management of population-based prescribing can be entered on the ledger/blockchain as well to allow for additional evaluation of the prescribing pharmacies in an area or prescribers in the area to look for patterns of unusual prescribing behavior and thus enable few narcotic prescriptions, if necessary. That is, the AI can track the prescribing, dispensing, and patient use patterns in defined regions.

In certain embodiments, the dispenser includes a secure element that stores a device keypair. A smart contract verifies a threshold signature (e.g., device key+prescriber key) before emitting an UnlockAuthorized event. Firmware transitions the lock state only upon successful verification of a contract-emitted event carrying a Merkle-authenticated dispenser state root that matches locally hashed sensor data. This event-driven, authenticated state transition reduces spoofing of dispensing events relative to conventional server-polled dispensers and improves integrity of access control in intermittently connected environments.

In some embodiments, the dispenser can cache a contract-issued authorization token that includes an expiry, a nonce, and a commitment to the pre-dispense state (e.g., a hash of sensor readings and inventory). While intermittently offline, the control unit may unlock subject to local policy (e.g., capped per-dispense quantity, T_max for window duration, and a minimum inter-dispense interval). Upon reconnect, the control unit submits device-signed telemetry for reconciliation; tokens that are expired, replayed, or inconsistent with the committed state are rejected and may trigger an incident record. Offline allowances may be reduced or disabled based on risk score.

In certain embodiments, the present invention provides for a medication management system, comprising: (a) a distributed ledger network executing at least one smart contract; and (b) a medication dispenser comprising a lock actuator, a weighing unit, a control unit, and a secure element storing a device private key; wherein the smart contract maintains tokenized dose entitlements for a patient account and, responsive to a dispense request, verifies policy constraints and a threshold of cryptographic authorizations including at least a prescriber signature and a dispenser attestation, and emits an authorization event that (i) is verifiable on-chain by any node of the network and (ii) carries a hash commitment to a pre-dispense dispenser state; wherein the control unit, prior to unlocking, authenticates the authorization event using a public key infrastructure and a device certificate bound to the secure element, verifies that the committed pre-dispense state matches locally measured sensor data, and transitions the lock actuator from a locked state to an unlocked state only upon successful verification; wherein during a dispense window the weighing unit measures a mass change corresponding to a target dose and the control unit generates a telemetry packet including a nonce, timestamps, pre-/post-dispense mass, and a tamper flag, signs the telemetry packet with the device private key, and causes submission of the telemetry packet to the smart contract via an authenticated oracle; and wherein the smart contract rejects stale or replayed telemetry by nonce or timestamp policy and commits a Merkle-authenticated digest of accepted telemetry to the distributed ledger, updates the tokenized dose entitlements, and records any reconciliation of entitlements pursuant to policy.

In certain embodiments, the blockchain network is permissioned and uses a Byzantine-fault-tolerant ordering service, and the smart contract enforces multi-signature authorization requiring at least a prescriber key and a device key.

In some embodiments, the dispenser includes a secure element storing a device private key, and the smart contract validates a dispenser-signed telemetry packet prior to authorizing dispensing.

In some embodiments, the patient-identifying data are stored off-chain, and on-chain records store only salted cryptographic digests that enable verification without revealing protected health information.

In some embodiments, the tokens are non-fungible tokens that encode a package identifier, dose count, and MME normalization constant and are burned upon verified return or destruction of unused medication.

In some embodiments, the emergency override requires threshold signatures from at least two of: prescriber, pharmacist, and caretaker, and generates an immutable incident report.

In some embodiments, the weighing unit samples at a defined cadence and the control unit transitions between LOCKED, AUTH_PENDING, DISPENSE, and AUDIT states responsive to smart-contract events.

In some embodiments, the AI module outputs a risk score and the smart contract applies parameterized rate-limits to a maximum MME per rolling period based on the risk score.

In certain embodiments, the system further comprising a digital twin of the dispenser maintained by the smart contract as a state object keyed to a dispenser identifier, the digital twin comprising state variables including at least inventory mass, calibration parameters, last authorization event identifier, entitlement balance, and a tamper status, wherein the smart contract computes and stores a hash commitment to the digital twin state in the distributed ledger.

In other embodiments, the authorization event includes a commitment to a predicted post-dispense twin state, and the control unit refuses to transition the lock actuator to the unlocked state unless a difference between the locally measured pre-dispense inventory and the digital twin's committed pre-dispense inventory is within a tolerance c.

In other embodiments, the acceptance of a telemetry packet causes the smart contract to advance the digital twin by applying the reported mass delta and tamper status, recomputing a twin state commitment, and emitting an event if the recomputed commitment deviates from a previously predicted commitment by more than a policy-defined threshold.

In other embodiments, the smart contract records a divergence incident and requires elevated authorization if the digital twin predicts a remaining inventory that differs from the weighing unit by more than F over N consecutive dispenses.

In other embodiments, the digital twin supports a simulation mode in which proposed entitlement changes or dosing schedules are evaluated off-chain and committed on-chain only as a state commitment and summary metrics, without disclosing protected health information.

In one embodiment, a per-dispenser digital twin is versioned and anchored by recording a digest of the twin state after each accepted dispense. The twin state may include (by way of example) the pair (mass_before, mass_after), computed delta, environmental conditions, the applicable policy version, and a sequence counter N. Divergence between the twin and locally measured state beyond F constitutes a reconciliation exception and can trigger audit, rate limiting, or a requirement for additional authorization. Upon successful reconciliation, the twin advances to version N+1, and a commitment to the updated twin is recorded (e.g., on-chain or in a hash-addressed store referenced by the chain).

In certain embodiments, the present invention provides for a computer-implemented method of controlling a medication dispenser using a distributed ledger, comprising: maintaining, by a smart contract, tokenized dose entitlements; verifying, responsive to a dispense request, policy constraints and threshold authorizations; emitting, by the smart contract, an authorization event that commits to a pre-dispense dispenser state; authenticating, by a control unit of the dispenser, the authorization event and verifying the committed state against locally measured sensor data; unlocking a lock actuator only upon successful verification; measuring a mass change during a dispense window; generating and device-signing a telemetry packet including a nonce, timestamps, pre-/post-dispense mass, and a tamper flag; submitting the telemetry packet via an authenticated oracle; and committing, by the smart contract, a Merkle-authenticated digest of accepted telemetry to the distributed ledger while updating the tokenized dose entitlements and rejecting stale or replayed telemetry by nonce or timestamp policy.

The disclosed architecture improves the operation of medication dispensers and the integrity of access control by cryptographically binding unlock authorization to a measured, device-attested dispenser state and by committing Merkle-authenticated telemetry on-chain. Unlike conventional server-polled systems, the control unit refuses unlock unless a contract-emitted authorization event is authenticated and the committed pre-dispense state matches locally hashed sensor data; stale/replayed telemetry is rejected by nonce/timestamp policy. These mechanisms produce a tamper-evident audit trail and reduce diversion with measurable, device-level guarantees.

Historically, management of the total amount of narcotics prescribed that the industry of pharmaceuticals has not been performed well by the DEA, government, or by any population standards. The present invention allows for AI to manage all aspects of narcotics production, prescription, dispensing, and use and to look for patterns and predict outcomes. Monetization allows for patient incentivization, tax options for management of narcotic use and prescribing patterns.

### BRIEF DESCRIPTION OF THE DRAWINGS

FIG. 1 exemplarily illustrates an environment of a blockchain based system for medication management and incentivizing medication management, according to an embodiment of the present invention.

FIG. 2 exemplarily illustrates a block diagram of a medication dispenser, according to an embodiment of the present invention.

FIG. 3 exemplarily illustrates a block diagram of a hardware module, according to an embodiment of the present invention.

FIG. 4 exemplarily illustrates a flowchart of a blockchain based method for medication management and incentivizing medication management, according to an embodiment of the present invention.

### DETAILED DESCRIPTION OF EXAMPLE EMBODIMENTS

### Definitions

As used herein, unless the context clearly indicates otherwise, the following terms have the meanings set forth below. The singular includes the plural and vice-versa; “or” is inclusive; and “comprise,” “comprising,” “include,” and “including” are open-ended. Numerical ranges include their endpoints and intermediate values. “About” or “approximately” with respect to a stated value means within a reasonable manufacturing or measurement tolerance (e.g., ±10% unless otherwise specified).

“Authorization event” means a ledger-recorded, smart-contract-emitted message (e.g., a log/event) that signals approval to transition a dispenser from a locked to an unlocked state for a specified request. The authorization event includes one or more of: a unique nonce, an expiry or valid-from/valid-to interval, policy parameters (e.g., maximum MME per window), and a cryptographic commitment (e.g., a hash) to a pre-dispense dispenser state and/or entitlement state. The control unit authenticates the authorization event prior to unlocking.

“Current period” means the evaluation window used to compare a request against the predefined limit, which may be a fixed calendar period (e.g., a day, week, or month), a sliding or rolling window (e.g., the most recent 24 hours), or a policy-defined interval keyed to a prior event (e.g., last dispense time).

“Dispense window” means a finite interval following an authorization event during which the dispenser is permitted to unlock and dispense. The window opens upon receipt and successful verification of the authorization event and closes upon the earliest of: (i) measurement of a mass change corresponding to the target dose within a tolerance F; (ii) lapse of a maximum duration Tₘₐₓ; or (iii) detection of a tamper event or policy violation.

“Epsilon (F) tolerance” means a numerical tolerance used by the control unit and/or verification logic when comparing a measured quantity (e.g., mass delta from a weighing unit) to an expected quantity. F may be expressed as an absolute value, a percentage, or a function of environmental/sensor conditions, and may be fixed or adaptively learned.

Herein, the term “distributed ledger” is intended to be broadly construed as a form of ledger data structure that can be shared, in whole or in part, among multiple computing or storage nodes. Example distributed ledgers technologies that could be used with the disclosed subject matter include blockchains, smart contracts, Openchain techniques, IOTA related directed acyclic graph techniques, hashgraph-utilizing techniques, hash chain-utilizing techniques, or hash-chain/Merkle-DAG structures, or other ledger based data structure technologies. It may not be necessary to completely distribute all data in the ledger. In fact, for some cases, an event and its associated ledger data structure can be held on a locked-down system, and it may be sufficient to log data on a single computing system. This may be done to preserve space on servers or bandwidth on a network so as not to interfere with the real-time nature of an event.

“Entitlement” means a tokenized, machine-verifiable right to receive a medication dose under defined constraints. An entitlement may be fungible or non-fungible and may encode metadata such as package identifier, strength, count, MME normalization constant, expiry, prescriber identity, dispensing cadence, and policy constraints. Entitlements are decremented, reconciled, burned, or otherwise updated by the smart contract upon acceptance of authenticated telemetry.

As used herein, the term “ledger data structures” is intended to convey the concept of units of digital data linked together to form a ledger of information. Examples of such data structures can include blockchains, hashgraphs, directed acyclic graphs (DAG), linked lists, or other forms of linked structures. Further, ledger data structures can be distributed among multiple computing nodes, can be centralized, or a combination of both. In more preferred embodiments, the ledger data structures further include a form of digital notarization as discussed further below.

“Merkle-authenticated digest” means a cryptographic commitment (e.g., a Merkle root) computed over a set of records such that membership and integrity proofs for individual records can be verified efficiently against the committed root stored on the ledger.

“Morphine Milligram Equivalents” or “MME” means a normalization of opioid dose magnitude to an equivalent mass of morphine over a defined period. In certain embodiments, MME for a rolling 24-hour window is computed as:

MME(24h)=Σᵢ(dose_massᵢ[mg]×conversion_factorᵢ),

where conversion_factorᵢ is a policy-defined constant for the active agent and route of administration. Unless specified otherwise, dose_mass; is the dispensed amount measured by the weighing unit and verified by telemetry. The conversion_factor used in MME computation is provided by a policy table keyed at least by active agent and route of administration (e.g., oral, transdermal), with optional fields for formulation and release profile. The policy table is versioned and may be anchored on-chain (e.g., by recording a root digest) or maintained off-chain with a hashed commitment to enable independent verification. Dose evaluation applies the current table version and is performed over a rolling window that defines the current period (e.g., a sliding 24-hour window), such that requests are compared against predefined limits expressed in MME or absolute quantity within the applicable current period.

“Nonce” means a value intended to be used once to ensure uniqueness of a request, authorization event, or telemetry packet. A nonce may be random, pseudorandom, a counter, or a value derived from contextual data, and is scoped at least to a device, session, or authorization event to prevent replay.

“Oracle” means an authenticated service that relays data between off-chain components (e.g., the dispenser or gateway) and on-chain smart contracts, while preserving integrity, authenticity, and replay protection.

“Patient wallet” means a cryptographically controlled account associated with a patient, implemented on a distributed ledger and controlled by one or more public/private keypairs. The patient wallet may be custodial or non-custodial, may support multi-signature or threshold authorization, and is configured to hold, receive, or spend tokenized dose entitlements and to receive contract-emitted events relevant to dispensing.

“Policy graph” refers to a data structure identifying permitted principals (e.g., users, devices, organizations, smart-contract addresses) and allowed relationships among them for issuing, updating, or reconciling dose entitlements. In one embodiment, the policy graph encodes allowed transfer or modification paths as edges; transactions targeting nodes not present in the graph are rejected. The policy graph may be versioned and its current state may be cryptographically committed (e.g., by a root digest recorded on-chain).

“Predefined limit” means a set of policy parameters that constrain dispensing, which may include, without limitation: a maximum total quantity (e.g., per 24-hour period), a per-dispense maximum, a minimum inter-dispense interval, and/or a maximum morphine milligram equivalent (MME). A predefined limit may be static, role-based, risk-adaptive, drug-specific, and/or time-varying.

A “private key” means a secret cryptographic key associated with an asymmetric key pair (e.g., ECDSA, EdDSA, RSA) that is used to generate digital signatures and/or decrypt messages. In preferred embodiments, the private key is generated and stored within a secure element or comparable hardware security module and is not exportable. Private keys may be rotated or revoked according to policy.

“Reconciliation” means any on-chain update of entitlement balances or states to reflect unused, returned, expired, or revoked doses, which may include decrement, burn, reissue, or credit under policy without implying monetary exchange.

“Risk score” means a scalar or vector value generated by a rules engine and/or machine-learning model that characterizes adherence or diversion risk for a subject, device, or transaction. Inputs may include historical usage, adherence metrics, anomaly signals from sensors, clinician annotations, and/or external data. The risk score may parameterize one or more predefined limits, require additional authorization (e.g., multi-party approval), or drive audit frequency.

“Secure element” means a hardware component provisioned with a device private key and supporting secure storage, cryptographic operations, and attestation used to authenticate dispenser messages and verify contract-emitted events.

“Tamper event” means a condition indicating possible physical interference with the dispenser or its contents, detected by one or more sensors (e.g., enclosure switch, accelerometer, tilt, seal integrity, unexpected lid open, anomalous mass delta while locked). In some embodiments a tamper event sets a sticky flag that persists until cleared under authenticated procedure and may inhibit dispensing or require elevated authorization.

“Telemetry packet” means a structured, device-signed record generated by the control unit that includes one or more of: a nonce, timestamps, pre- and post-dispense mass values, computed mass delta, tamper flag(s), device identifier, firmware version, and optional diagnostics. Accepted telemetry packets are validated (e.g., signature, freshness) and committed to the ledger directly or via an oracle.

“Threshold (Multi-Party) Authorization” means an access control policy that requires cryptographic approval from at least a specified subset (e.g., t-of-n) of distinct principals (e.g., prescriber, pharmacist, caretaker, device) before a smart contract emits an authorization event.

A “timestamp” is a time indicator associated with a message or event, which may represent wall-clock time, monotonic time, or both. A timestamp may be obtained from a device clock, a trusted time source, or an authenticated oracle and may be included in signed data to support ordering, expiry, and window enforcement.

“T_max” or “maximum dispense window duration” means a policy parameter defining the maximum time allowed between an authorization to unlock and a required re-lock or dispense completion. Exceeding T_max may trigger an abort, a tamper event, or a requirement for renewed authorization.

Example embodiments of the disclosure now will be described more fully hereinafter with reference to the accompanying drawings, in which example embodiments are shown. The concepts discussed herein may, however, be embodied in many different forms and should not be construed as limited to the example embodiments set forth herein; rather, these embodiments are provided so that this disclosure will be thorough and complete, and will fully convey the scope to those of ordinary skill in the art. Like numbers refer to like elements but not necessarily the same or identical elements throughout.

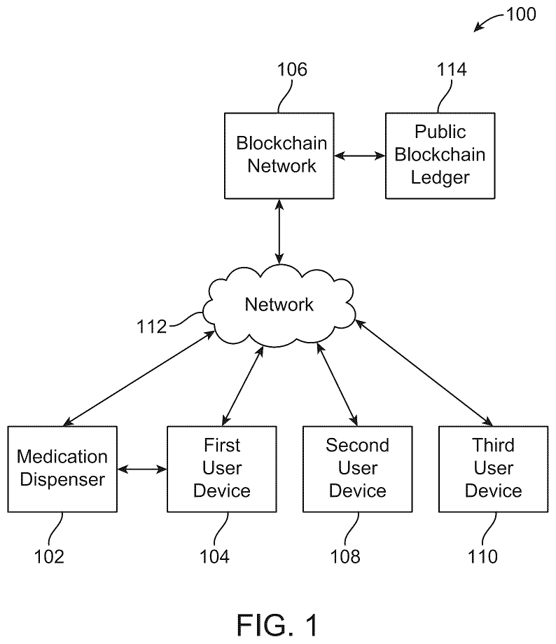

Referring to FIG. 1, the environment 100 comprises a medication dispenser 102, a first user device 104 and a blockchain network 106. The medication dispenser 102 and the first user device 104 are associated with a first user. The environment 100 further comprises a second user device 108 associated with a second user and a third user device 110 associated with a third user. For example, the first user is a patient, the second user is a healthcare provider and the third user is an administrator. In one embodiment, the administrator could be a pharmacy, drug manufacturer or any other entity.

In another embodiment, the management administrator is an artificial intelligence (AI) or artificial general intelligence (AGI) system. In another embodiment, the management administrator is a governmental agency (e.g., the Drug Enforcement Agency) for both management and enforcement.

The blockchain network 106 can be comprised of a plurality of decentralized computing (blockchain) nodes. Each blockchain node can be a computing system, such as illustrated in the figures, that is configured to perform functions related to the processing and management of the blockchain, including the generation of blockchain data values, verification of proposed blockchain transactions, verification of digital signatures, generation of new blocks, validation of new blocks, and maintenance of a copy of the blockchain. In one embodiment, the blockchain network comprises a decentralized blockchain with contract functionality (e.g., Ethereum).

The blockchain can be a distributed ledger that is comprised of at least a plurality of blocks. Each block can include at least a block header and one or more data values. Each block header can include at least a timestamp, a block reference value, and a data reference value. The timestamp can be a time at which the block header was generated and can be represented using any suitable method (e.g., UNIX timestamp, DateTime, etc.). The block reference value can be a value that references an earlier block (e.g., based on timestamp) in the blockchain. In some embodiments, a block reference value in a block header can be a reference to the block header of the most recently added block prior to the respective block. In an exemplary embodiment, the block reference value can be a hash value generated via the hashing of the block header of the most recently added block. The data reference value can similarly be a reference to the one or more data values stored in the block that includes the block header. In an exemplary embodiment, the data reference value can be a hash value generated via the hashing of the one or more data values.

The use of the block reference value and data reference value in each block header can result in the blockchain being immutable. Any attempted modification to a data value would require the generation of a new data reference value for that block, which would thereby require the subsequent block's block reference value to be newly generated, further requiring the generation of a new block reference value in every subsequent block. In some embodiments, the blockchain can be used to store information regarding blockchain transactions conducted between two different blockchain wallets. A blockchain wallet can include a private key of a cryptographic key pair that is used to generate digital signatures that serve as authorization by a payer for a blockchain transaction, where the digital signature can be verified by the blockchain network 106 using the public key of the cryptographic key pair. In some cases, the term “blockchain wallet” can refer specifically to the private key. In other cases, the term “blockchain wallet” can refer to a computing device (e.g., a computing device, etc.) that stores the private key for use thereof in blockchain transactions. For instance, each computing device can each have their own private key for respective cryptographic key pairs and can each be a blockchain wallet for use in transactions with the blockchain associated with the blockchain network. Computing devices (104, 108, 110) can be any type of device suitable to store and utilize a blockchain wallet, such as a desktop computer, laptop computer, notebook computer, tablet computer, cellular phone, smart phone, smart watch, smart television, wearable computing device, implantable computing device, etc.

The medication dispenser 102, the first user device 104, the second user device 108 and the third user device 110 are in communication with the blockchain network 106 via a network 112. In one embodiment, the first user device 104, the second user device 108 and the third user device 110 are directly in communication with the blockchain network 106. This is accomplished through a “RPC node” using an API. The first user device 104, the second user device 108, and the third user device 110 also referred as user device (104, 108, 110).

In one embodiment, the user device (104, 108, 110) includes at least one of a mobile phone, a tablet computer, a laptop computer, a desktop computer, a wearable computer, and/or any other suitable type of computer. In another embodiment, the user device (104, 108, 110) includes media playback devices, such as a television, speakers, a game console, and/or any other suitable type of media playback device. The user device (104, 108, 110) further includes any suitable Internet of Things (IoT) devices.

The network 112 could be any suitable combination of one or more wired and/or wireless networks in some embodiments. For example, network 112 includes any one or more of the Internet, an intranet, a wide-area network (WAN), a local-area network (LAN), a wireless network, a digital subscriber line (DSL) network, a frame relay network, an asynchronous transfer mode (ATM) network, a virtual private network (VPN), and/or any other suitable communication network. The user device (104, 108, 110) is connected by one or more communications links to communication network 112 that can be linked via one or more communications links to the blockchain network 106. In some embodiments, the user device (104, 108, 110) could be connected to the communication network 112 via one or more routers. In some embodiments, the communications links can be any communications links suitable for communicating data among user devices (104, 108, 110) and blockchain network 106 such as network links, dial-up links, wireless links, hard-wired links, any other suitable communications links, or any suitable combination of such links.

The blockchain network 106 stores information, data, programs, and/or any other suitable content. Although the blockchain network 106 is illustrated as one device, the functions performed by blockchain network 106 can be performed using any suitable number of devices. In one embodiment, multiple devices are used to implement the functions performed by blockchain network 106. In one embodiment, the blockchain network 106 is a cloud blockchain network. The blockchain network 106 includes at least one of q public, a permissioned, and a hybrid (public/private) blockchain.

The blockchain network 106 includes one or more processors and one or more memories. The memory comprises a set of program modules. The program modules are executed by the processor to perform medication management and incentivizing medication management. The blockchain network 106 comprises a distributed ledger/blockchain protocol 114 to store information related to medication management and incentivizing medication management. In one embodiment, the distributed ledger/blockchain protocol 114 is a part of blockchain of the blockchain network 106. The blockchain network 106 in communication with the medication dispenser 102 configured to receive information related to the functions being executed at the dispenser 102. The medication dispenser 102 is linked to the first user device 104 via an authentication system. In an embodiment, the authentication system includes at least one of a biometric scan-based authentication system and a two-factor authentication system. The authentication system also includes any other additional electronic authentication or third party authentication. The authentication system also includes multi-factor authentication.

In one embodiment, the blockchain network 106 comprises a network of computing nodes (also referred as computer network). Each computing node comprises a memory and a processor configured to execute instructions stored on the processor. In one embodiment, the network will be permissioned and allows certain user devices (104, 108, 110) and computing nodes to join the network In one embodiment, the computer network comprises a smart contract stored on the memory of each computing node of the plurality of computing nodes. The smart contract may execute resource transactions and smart contract instructions.

As described herein, a “smart contract” is a computer program that is capable of automating execution of the terms of a machine-readable contract based on rules that can process inputs in order to produce results, which can cause actions to be performed that are dependent on these results. Generally, smart contracts are used in the transfer of property rights or assets. In general, a “smart contract” is a machine-executable program deployed to a distributed ledger that defines deterministic state-transition functions and business rules, such that, upon receiving valid inputs (e.g., transactions, messages, or events), the contract logic is executed by validator/endorser nodes and the resulting state updates are committed to the ledger. Smart contracts may be implemented on public, private, or hybrid/consortium blockchains and may interoperate with off-chain services via authenticated calls or oracles. In some embodiments, the contract is compiled to a platform-specific bytecode (e.g., Ethereum Virtual Machine bytecode) and executed deterministically by multiple nodes with metered resource usage (e.g., gas accounting), emitting logs/events and persisting data to on-chain key-value state (e.g., Merkle-authenticated stores). Upgrades can be supported through versioned deployments or proxy patterns while preserving contract addresses/state. By way of example and not limitation, suitable platforms include Hyperledger Fabric, in which smart contracts are implemented as “chaincode” executed by endorsing peers per an endorsement policy and ordered via a pluggable ordering service; R3 Corda, in which contracts and associated flows govern peer-to-peer state evolutions under notary consensus without global broadcast; Hyperledger BESU, an enterprise Ethereum client supporting permissioned or permissionless EVM-compatible networks (e.g., with Solidity/Vyper contracts); and Stacks, which anchors transactions to Bitcoin and executes smart contracts written in Clarity for predictable, decidable behavior. The foregoing platforms are illustrative of environments in which the disclosed smart-contract functionality can be realized, and any equivalent ledger capable of deterministic contract execution and consensus-based state commitment may be used.

In another embodiment, the blockchain network 106 is a Ethereum blockchain network maintained by a network of computing nodes. Each computing node comprises a memory and a processor configured to execute instructions stored on the processor. In one embodiment, the network will be permissioned and allows certain user devices (104, 108, 110) and computing nodes to join the network. The computer network comprises a smart contract which is software code residing on the blockchain of each computing node of plurality of computing nodes, which is designed to automatically carry out specific tasks if certain conditions are met. The smart contract may execute resource transactions and instructions. In one embodiment, the smart contract operates on the Ethereum blockchain network and executes transactions, known as ether or ETH powered transactions, as resource transactions and implements the software instructions of smart contracts submitted to the Ethereum blockchain network. These computing nodes collectively validate and confirm transactions, maintain a copy of the blockchain's entire history, and participate in the consensus mechanism to agree on the state of the network. The Ethereum blockchain network comprises a decentralized and distributed ledger that stores a record of all transactions and smart contracts executed on the Ethereum blockchain network. As the system is run as a private blockchain, the Ethereum blockchain network enables to control the number of confirmations needed to validate a transaction.

The network of computing nodes runs Ethereum client software. Ethereum client software is used to connect to the Ethereum blockchain network, send transactions, deploy and execute smart contracts, and retrieve blockchain data. This software acts as a gateway for users to interact with the Ethereum blockchain and utilize its capabilities. Ethereum client software enable users to interact with the Ethereum blockchain network.

In another embodiment, the system is configured to support various blockchain platforms, including, but not limited to, Ethereum, Bitcoin, Solana, Cardano, Ripple, BNB Chain, and potentially other emerging blockchain platforms. The system facilitates deployment and execution of smart contracts across multiple blockchain networks, providing flexibility and adaptability to changing technological landscapes. The system provides flexibility to deploy smart contracts on public, private, and hybrid blockchains, which enables to execute diverse use cases. By widening the scope to encompass various blockchain platforms, the present invention ensures versatility and future-proofing in the field of decentralized applications and smart contract execution.

In yet another embodiment, the system of the present invention could be implemented as a digital twin system. The digital twin represents the medication dispenser 102 in the virtual environment. It maintains real-time synchronization with the physical dispenser 102, capturing data such as medication quantities, refill status, and dispensing activity. The first user device 104 and second user device 108 are also represented as digital twins. These virtual representations enable interaction with the blockchain network 106 and facilitate communication between users and the medication dispenser digital twin.

The blockchain network 106 serves as the backbone of the digital twin system, providing a secure and decentralized platform for managing medication-related transactions and incentives. Smart contracts deployed on the blockchain govern interactions between digital twins and users, ensuring transparency, immutability, and trust. In the digital twin system, the second user (represented by their digital twin) can prescribe a quantity of medication to the first user (also represented by their digital twin) through interactions with the blockchain network 106. The corresponding tokens are transferred between the digital twins to incentivize medication management. Upon receiving approval from the blockchain network 106, the medication dispenser digital twin dispenses medication to the first user's digital twin. The tokens assigned to the first user are updated based on the remaining quantity of medication at the dispenser, reflecting real-world usage. The first user's digital twin can trade unused tokens for a monetary value with the guidance of the second user's digital twin. This exchange of tokens for monetary value is facilitated through smart contracts on the blockchain, ensuring transparency and security.

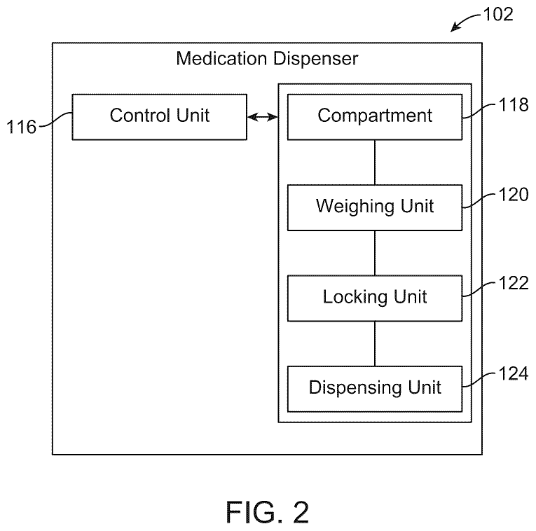

Referring to FIG. 2, the medication dispenser 102 comprises at least one compartment 118, a control unit 116, a weighing unit 120, a locking unit 122 and a dispensing unit 124. The control unit 116 is in communication with the weighing unit 120, the locking unit 122 and the dispensing unit 124. The control unit 116 is configured to control one or more functions of the weighing unit 120, the locking unit 122 and the dispensing unit 124. The compartment 118 is configured to store a quantity of medication. In another embodiment, the medication dispenser 102 comprises a plurality of compartments 118. Each compartment 118 is configured to store different medications. The medication dispenser 102 is in communication with the first user device 104 via the network 112, for example, Bluetooth.

The weighing unit 120 is configured to transmit information related to the quantity of medication in the compartment 118 to the blockchain network 106. In one embodiment, the weighing unit 120 comprises a weighing sensor. The locking unit 122 is configured to lock the dispensing unit 124 to prevent dispense of medications without authorization. The control unit 116 is configured to enable the locking unit 122 to unlock the dispensing unit 124 based on the approval of the request received from the first user. The control unit 116 is configured to enable the dispensing unit 124 to dispense the requested quantity of medication.

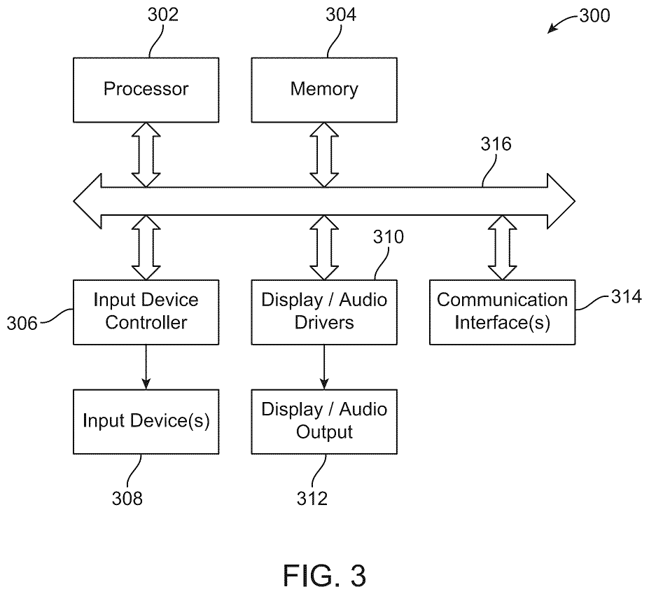

Referring to FIG. 1, in one embodiment, the blockchain network 106 and user devices (104, 108, 110) can be implemented using any suitable hardware. For example, the device (106, 104, 108, 110) could be implemented using any suitable general-purpose computer or special-purpose computer. For example, a mobile phone may be implemented using a special-purpose computer. Any such general-purpose computer or special-purpose computer can include any suitable hardware. For example, as illustrated in example hardware module 300 of FIG. 3. Referring to FIG. 3, the hardware module 300 includes hardware processor 302, memory 304, an input device controller 306, an input device 308, display/audio drivers 310, display and audio output circuitry 312, communication interface(s) 314, and a bus 316.

The processor 302 includes any suitable hardware processor, such as a microprocessor, a micro-controller, digital signal processor(s), dedicated logic, and/or any other suitable circuitry for controlling the functioning of a general-purpose computer or a special-purpose computer. In one embodiment, the processor 302 is controlled by a server program or program modules stored in memory 304 of the blockchain network 106. For example, in some embodiments, the program modules at the blockchain network 106 are configured to cause the processor 302 to add a block to the blockchain, synchronize a cloud blockchain with a blockchain locally stored on a user device (104, 108, 110), and/or perform any other suitable functions. In one embodiment, the processor 302 is controlled by a computer program stored in memory 304 of the user device (104, 108, 110). For example, the program modules cause the processor 302 to perform medication management and incentivizing medication management.

The memory 304 includes any suitable memory and/or storage for storing programs, data, and/or any other suitable information. In one embodiment, memory and/or storage 304 include random access memory, read-only memory, flash memory, hard disk storage, optical media, and/or any other suitable memory. The input device controller 306 includes any suitable circuitry for controlling and receiving input from one or more input devices 308. For example, the input device controller 306 could be circuitry for receiving input from a touchscreen, from a keyboard, from one or more buttons, from a voice recognition circuit, from a microphone, from a camera, from an optical sensor, from an accelerometer, from a temperature sensor, from a near field sensor, from a pressure sensor, from an encoder, and/or any other type of input device.

The display/audio drivers 310 include any suitable circuitry for controlling and driving output to one or more display/audio output devices 312. For example, display/audio drivers 310 include circuitry for driving a touchscreen, a flat-panel display, a cathode ray tube display, a projector, a speaker or speakers, and/or any other suitable display and/or presentation devices. The communication interface(s) 314 include any suitable circuitry for interfacing with one or more communication networks (e.g., network 112). For example, interface(s) 314 includes network interface card circuitry, wireless communication circuitry, and/or any other suitable type of communication network circuitry. Bus 316 includes any suitable mechanism for enabling communication between two or more components 302, 304, 306, 310, and 314.

Referring to FIG. 1 and FIG. 2, the distributed ledger is a blockchain network 106 that utilizes smart contracts, or other related contracting mechanisms capable of supporting and executing self-regulating computer program or a transaction protocol that is intended to automatically execute, control or document events and actions according to specified terms. In some embodiments, the smart contracts are configured to perform one or more functions. Further, each function and/or steps executed by the blockchain network 106 is recorded for tracking/reporting purposes. The blockchain network 106 is configured to receive parameters related to medication for medication management and incentivizing medication management. The parameters include, but are not limited to, the medication name, medication dosage for a period of time, intervals between each medication dosage and medication related instructions. In addition, parameters may include practices of use that would include “as needed” usage (PRN) indicating a methodology that the patient has an ability to manage through use or nonuse pattern. This variance indicates patient use of medication outside of the regular dosage pattern of mandated use. The parameters are received from the second user or the healthcare provider via the second user device 108. Further, profile information related to the first user is received from the first user via the first user device 104. In another example, the profile information related to the first user is received from the first user under the supervisor of the healthcare provider. The profile information includes, but is not limited to, the first user's name, contact number, and information related to the biometrics of the first user to link the medication dispenser 102 and the first user device 104.

The blockchain network 106 is configured to enable the third user to allocate a predefined number of tokens for a predefined quantity of medication to the second user. Each token comprises a monetary value. The blockchain network 106 is further configured to enable the third user to transfer the tokens to the second user. The blockchain network 106 is configured to provide a virtual wallet to the first user and the second user. The first user and the second user could access the wallet via the respective user device (104, 108). The blockchain network 106 is configured to transfer the tokens from the third user to the virtual wallet of the second user. The blockchain network 106 is configured to enable the second user to prescribe a quantity of medication to be filled in the compartment 118 and transfer a number of tokens corresponding to the quantity of medication prescribed to the first user, via the second user device 108.

The blockchain network 106 is configured to enable the first user to raise a request to unlock the medication dispenser 102 and to dispense a desired quantity of medication, via the first user device 104. The first user raises a request to dispense medication via the first user device 104. The blockchain network 106 is further configured to receive a request from the first user, via the first user device 104, to dispense a quantity of medication.

On receiving the request from the first user, the blockchain network 106 is configured to retrieve at least one of an information corresponding to the quantity of medication stored after a previous dispense period and information corresponding to the quantity of medication present in the medication dispenser 102. The blockchain network 106 is further configured to retrieve the information related to the requested quantity of medication for a current period of time. Then, the blockchain network 106 is configured to determine if the requested quantity of medication is within a predefined limit for the current period of time. The blockchain network 106 is further configured to approve the request to dispense the requested quantity of medication when the requested quantity of medication is within the predefined limit for the current period of time. Further, the blockchain network 106 is configured to regularly check the quantity of medication in the compartment 118 after a predefined interval of time. Based on the regular check and the available tokens, the blockchain network 106 is configured to track the amount of medication left in the compartment 118.

The blockchain network 106 is further configured to record information related to the quantity of medication present in the dispenser 102 after each dispensing period and information related to the requests from the first user to dispense the quantity of medication in the distributed ledger/blockchain protocol 114. The blockchain network 106 is further configured to update the tokens assigned to the first user based on the dispensed quantity of medication. The blockchain network 106 is further configured to enable the first user to trade the tokens corresponding to the unused quantity of medication for a monetary value with the guidance of the second user. The exchange of tokens under the guidance of the second user prevents any counterfeiting activity. The tokens could be exchanged for cash and could be used in a trading platform.

Based on the usage of medication and tokens, the blockchain network 106 is configured to track the usage of medication. In one embodiment, the medication comprises opioid drugs. The opioid includes, but not limited to, liquid morphine, fentanyl, and Demerol® (meperidine). The system is configured to track medication usage in morphine-equivalent units. The used tokens are recycled from the system. The value of the token may change. Alternatively, the tokens used for exchange by the first user could be acquired by the second user and transmitted back to the third user.

The blockchain network 106 is further configured to create packets of information or alerts based on the usage of medication. In one embodiment, the blockchain network 106 is configured to send alert to the second user when the usage of the medication of the first user exceeds the predefined limit. In one embodiment, the second user is the prescribing physician such that medication changes may be made such as changes to the dosage regiments. In another embodiment, the second user is a controlling governmental agency. In another embodiment, the blockchain network 106 is configured to send alert to a caretaker when the usage of the medication of the first user exceeds the predefined limit. In yet another embodiment, the blockchain network 106 is configured to send alert to the first user. The blockchain network 106 is configured to create and store a usage history for each patient at the distributed ledger/blockchain protocol 114. The blockchain network 106 is configured to issue alerts or reports in the event of deviations in dispenser 102 weight from the expected value. The deviation of dispenser 102 weight could be determined using the weight sensor and usage history of medication. The blockchain network 106 is further configured to generates alerts or reports for unauthorized access or tampering. The blockchain network 106 is further configured to store and update patient data for each interaction. Each interaction contributes to the enhancement of the system's intelligence. The blockchain network 106 is further configured to provide an analysis and a report of available opioid drugs in the market and the quantity of opioid drugs produced for a predefined period of time.

For example, if the administrator transfers 100 tokens to the healthcare provider and the healthcare provider assigns and transfers 50 tokens to the patient. Initially, the virtual wallet of the patient comprises 50 tokens and after a period of time the patient has 10 number of tokens. The reduction in the number of tokens could be used to track the usage of medication of the first user. Since, the tokens are assigned by the healthcare provider and/or administrator, the system is configured to track usage in several levels, which include, from the administrator, to the healthcare provider and to the patient.

In one embodiment, a token represents a group of narcotics. In one embodiment, each token represents a specific number of narcotic equivalents. In one embodiment, token represents odd number of pills to increase difficulty of selling easily. In another embodiment, the token may represent a package of pills with dispenser. The dispenser could be electronically managed per pill. In one embodiment, the dispenser supports a stack of pills or linear pattern of pills. In another embodiment, the dispenser is a circular pattern of pills (pack). The Government with help of DEA defines total number of circulating and produced/destroyed packs in circulation.

Initially, a monetized token is sold to pharmaceutical company to allow production by the company. In one embodiment, any controlled substance can be used in the system although the amounts can vary depending on the enforcement agency. Once the drug is produced, the token is associated with the bar code or descriptor for the package of pills. This package is sold to the pharmacy with payment for the token and the pills. In one embodiment, the amount of narcotic equivalent is now increased in price because of the additional cost of the token and/or tax or other regulatory fees. Physician will create a prescription for a multiple of the package in narcotic pill equivalents. Then, patient obtains the prescription and then presents to the pharmacy. The pharmacy is able to compare the physician history and patient history against the current prescription. Physician history can be numerical against prior token use and prescribing history. Patient history can be numerical against prior token use and usage history. If numbers are below certain threshold determined by the DEA/State/Federal then prescription will be mobilized.

The payment of token by the patient (tax on the use of prescription) could be made possibly by an insurance company. The cost will be less to have additional limits on patient use of prescription medications. The system provides a patient portal for the token(s) created for home-based usage. The token usage history can determine educational needs for the patient in order to actuate the dispensing package to allow for use of the pills. Once package(s) are completely exhausted according to time of regular usage, the token is now eliminated. Unused pills can be returned and destroyed by the pharmacy. The token is then burnt, ensuring it remains out of circulation permanently. These tokens are directed to a wallet designated for receiving only. A smart contract may burn a token by transferring the token to an inaccessible address associated with a wallet or another smart contract (sometimes referred to as a “dead” or “invalid” address on the blockchain). Credit may be given to the patient to further prescription usage by the pharmacy for the unused portion of the packages.

In one embodiment, the insurance company can pay for the token as an additional advantage for use for patient care. If additional data is required for physician usage of tokens and patient history of tokens, then payment for this information is required and may pay for the system of token utilization.

The parties involved in the system includes patient, physician, hospital, pharmacy, pharmaceutical company and insurance company. The patient is enabled to use medication that is prescribed. The patient could use the least amount of medication to allow for additional credits from the pharmacy. The patient needs to define themselves as personally involved with their care with the insurance company to decrease their ultimate cost of insurance.

The system enables to prescribe medications to the patient and enable to monitor the patient for controlled use of the medications to decrease liability. Further, use of the system decreases Malpractice insurance. The token system could allow universal understanding of patient use of the system i.e., token number for the patient is their prescribing and usage history. Further, it enables to make informed decisions regarding practice of prescribing. The payments for higher use of narcotics i.e., pain management practice, requires payment into the token system. This is another way to monetize the system. Better practices could allow for referral from other physicians of patients for management. The hospital will act as the pharmacy in purchasing narcotic amounts from the pharmaceutical company or distribution company. These will similarly be regulated and tokenized per vial, per MG equivalent, per syringe or per pill package. Hospital outpatient pharmacy will be on the normal token system of use.

If pharmacy wants to provide medications, it will purchase token from the pharmaceutical company as additional money. This may incentivize them not to prescribe as much of the narcotic. Additional patient education to decrease usage of the pills and Management of higher use physicians can be reported through the system. The gatekeeper function of the pharmacy will be a significant issue of management of the token system although the system can have obvious blocks to use. If the pharmacy does not flag system when appropriate, they can be taxed additionally money. This could monetize the system and motivate the parties involved to increase patient and physician education.

If the pharmaceutical company wants to make medications, the system use will increase the cost to the company. Additional costs can be passed down to the pharmacy and may incentivize decrease in production of the controlled substances. Other companies may arise to manage these medications within the more limited and controlled use parameters. The data delivery for patient use of token will require additional money for each data delivery per patient and may be able to decrease the patient monthly cost of insurance as a shared risk assessment of health care. Data delivery for physician use of token will require additional money for each data delivery. The physician could receive incentives for good management of medication. It will Increase referrals from insurance and best practices defined for individual physicians. Overall costs will decrease and patient health will increase.

Token data management, based on, state usage, federal usage, physician best practices, patient usage patterns, decreases potential for addiction by using data for predictive model. The token usage patterns for any of the parties involved will be a twin determining the life cycle of the narcotic package. Token can be linked to usage information post operative, post injury, chronic care and oncology. Currently no real time management of use of narcotics. Current implemented system is OARRS, which helps to evaluate for history of use and prescribing for patient and for prescriber. The system enables to evaluate and manage narcotic problem. Insurance may be best management of use of token to allow more control of the patient and the physician practices. The system determines the blockchain owned and paid for by government, pharmaceutical company or insurance as this could change the parameters of use. The system further comprises artificial intelligence module, which influences data collection and regulation for the patient, physician, intervals of use, patterns of physician prescription, pharmaceutical dispensing and patient usage. The artificial intelligence module further involves in managing data of patient history, prior usage, socioeconomic data, case example data, police data. Moreover, constants derived from anticipated usage must be integrated into the operational framework of the system. Subsequently, each successive interaction with a patient will augment the dataset, thereby enhancing the system's intelligence over time, particularly in the context of managing narcotics.

The artificial intelligence module is configured to facilitate decision making concerning the prescription and access points of controlled substances. The system could be implemented at a wide range of entities, from national healthcare systems to private corporations and insurance providers, enabling comprehensive monitoring and management of narcotic usage.

The artificial intelligence module may implement a supervised gradient-boosted decision tree trained on features including: (i) MME-normalized daily dose, (ii) intra-day slope of weight-sensor readings, (iii) PRN variance score (a of inter-dose intervals), (iv) prior refill lag, (v) device tamper flag rate, and (vi) EHR comorbidity flags. The model outputs a risk score rϵ[0,1]. A smart contract enforces guardrails: if r≥r₁, require prescriber multi-sig to authorize dispensing; if r≥r₂>r₁, throttle dose to a capped MME per 24 h and trigger caretaker alerts with audit logs.

In certain embodiments, a risk-scoring “AI module” implements a supervised gradient-boosted decision tree model trained to predict non-compliant use or diversion. Training data comprise historical dispenser logs aligned to EHR-derived labels and clinician adjudications. Features include: (i) daily dose normalized to morphine-milligram equivalents (MME); (ii) intra-day slope and variance of weight-sensor readings; (iii) inter-dose interval irregularity (coefficient of variation); (iv) refill-lag and early-access attempts; (v) tamper-flag rate and duration; (vi) device connectivity gaps; and (vii) comorbidity and concurrent-therapy indicators. Features are z-normalized, missing values are imputed, and categorical inputs are one-hot encoded. The model outputs rϵ[0,1]. Thresholds are policy-set: if r≥r₁, the smart contract requires multi-signature authorization before emitting an authorization event; if r≥r₂>r₁, the contract rate-limits maximum MME per rolling period and triggers caretaker alerts. Training uses k-fold cross-validation with AUC/PR metrics; calibration employs Platt scaling or isotonic regression. Inference executes server-side or on a gateway, with a signed model artifact (version ID, hash) and a feature schema enforced at runtime. Concept drift is monitored by population-stability index; thresholds and model versions are dynamically updatable through governance transactions. Personally identifiable information is held off-chain; on-chain records store only salted digests and model outputs required for auditability.

In preferred embodiments, artifacts recorded to the distributed ledger are pseudonymous digests that omit protected health information (PHI). Subject identity and other regulated data are maintained in a segregated data store under role-based access control and attestation, with access events logged and periodically audited. On-chain entries reference the segregated records only via cryptographic commitments (e.g., salted digests or Merkle proofs), thereby enabling verification of integrity and ordering without disclosing PHI.

The system operates at the intersection of healthcare, technology, and data analytics, offering an effective approach to address concerns surrounding narcotic addiction, tolerance, and misuse. Through the integration of AI algorithms, the system analyzes patient data, including medical history, prior usage patterns, socioeconomic factors, and case examples, to inform prescription decisions and access point control.

The system of the present invention could be adapted to different scales of implementation. For instance, national healthcare systems could deploy the system to monitor and regulate narcotic usage on a population level, while private corporations and insurance providers could utilize it to manage prescriptions for their employees or members. Moreover, the system accommodates a diverse range of controlled substances, including opioids, benzodiazepines, Neurontin, and marijuana, among others.

The system provides a proactive approach to address opioid-related concerns within the medical community. By leveraging AI-driven decision making, the system aims to decrease the potential for addiction behaviors and mitigate the risk of narcotic epidemics. Furthermore, the system's dynamic nature allows it to evolve over time, incorporating real-time usage data to refine prescription guidelines and establish robust standards of care.

The system further comprises a dual verification mechanism, which serves as a unique identifier within a blockchain framework. This feature enables precise tracking and management of narcotic usage, enhancing transparency and accountability in the prescription process. The system and method provide a new standard for prescription management and access point control in the modern healthcare landscape.

In one embodiment, the dual verification combines a non-fungible token (NFT) with a digital payment instrument. A user may have an exclusive digital payment instrument, such as a digital credit card, which can prove ownership of both a transaction account for which the digital payment instrument is requested and an NFT that corresponds to the narcotic equivalent that is being obtained. Ownership or authorization of the transaction account can be performed using a suitable authentication process through, for instance, providing unique account information or login credentials for an account registered with an associated entity. Ownership or authorization of the NFT can be performed via validation of a digital signature generated using a blockchain wallet that has ownership of the NFT on a blockchain.

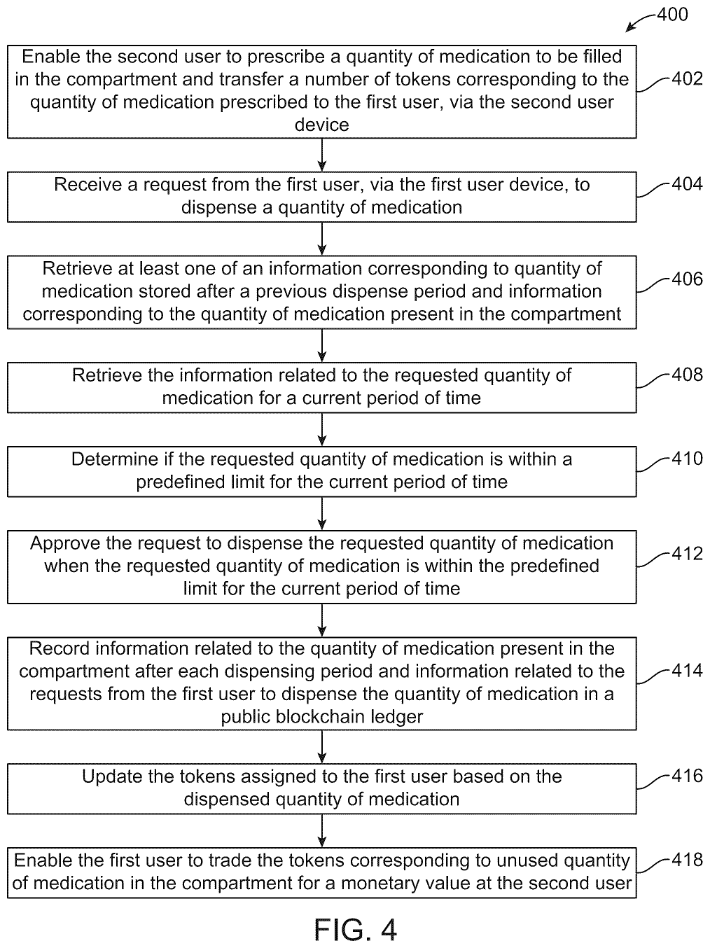

Referring to FIG. 4, the present invention discloses a method 400 for medication management and incentivizing medication management. The method is executed in a system comprising the medication dispenser 102, the first user device 104 and the blockchain network 106. The medication dispenser 102 and the first user device 104 are associated with the first user. The system further comprises the second user device 108 associated with the second user and the third user device 110 associated with the third user. For example, the first user is a patient, the second user is a healthcare provider and the third user is an administrator.

The medication dispenser 102, the first user device 104, the second user device 108 and the third user device 110 is in communication with the blockchain network 106 via the network 112 or via an RCP node. The first user device 104, the second user device 108 and the third user device 110 also referred as user device (104, 108, 110). The medication dispenser 102 further comprises at least one compartment 118, the control unit 116, the weighing unit 120, the locking unit 122 and the dispensing unit 124. The control unit 116 is in communication with the weighing unit 120, the locking unit 122 and the dispensing unit 124. The control unit 116 is configured to control one or more functions of the weighing unit 120, the locking unit 122 and the dispensing unit 124. The compartment 118 is configured to store a quantity of medication.

The weighing unit samples at ≥10 Hz with 0.01 g resolution and computes a rolling median to reject motion artifacts. A tamper channel (reed switch+accelerometer) sets a sticky ‘tamper’ bit if |Δa|>0.5 g for >200 ms while locked. The control unit implements states {LOCKED, AUTH_PENDING, DISPENSE, AUDIT}. A DISPENSE window closes automatically when |Δmass|≥dose_target±ε, where ε is a calibration tolerance (e.g., 0.05 g), or upon expiry Tₘₐₓ. The firmware emits a signed telemetry packet <nonce, pre/post mass, tamper, ts> to the smart contract via an oracle; the contract rejects stale or replayed packets by nonce.

At step 402, the blockchain network 106 enables the second user to prescribe a quantity of medication to be filled in the compartment 118 and transfer a number of tokens corresponding to the quantity of medication prescribed to the first user, via the second user device 108. The blockchain network 106 is configured to enable the third user to allocate a predefined number of tokens for a predefined quantity of medication to the second user. Each token comprises a monetary value. The blockchain network 106 is further configured to enable the third user to transfer the tokens to the second user. The third user assigns and transfers the token via the third user device 110.

At step 404, the blockchain network 106 receives a request from the first user, via the first user device 104, to dispense a quantity of medication from the medication dispenser 102. Generally, the dispensing unit 124 locks the compartment 118 to prevent dispensing of medication without authorization. The locking unit 122 is configured to lock the dispensing unit 124 to prevent dispensing of medication without authorization. At step 406, the blockchain network 106 retrieves at least one of an information corresponding to the quantity of medication stored after a previous dispense period and information corresponding to the quantity of medication present in the medication dispenser 102.

At step 408, the blockchain network 106 retrieves the information related to the requested quantity of medication for a current period of time. At step 410, the blockchain network 106 is configured to determine if the requested quantity of medication is within a predefined limit for the current period of time. At step 412, the blockchain network 106 approves the request to dispense the requested quantity of medication when the requested quantity of medication is within the predefined limit for the current period of time. The locking unit 122 in communication with the control unit 116 unlocks the dispensing unit 124 based on the approval of the request received from the blockchain network 106. Thereafter, the control unit 116 is configured to enable the dispensing unit 124 to dispense the requested quantity of medication. The weighing unit 120 comprises the weight sensor configured to transmit information related to the quantity of medication in the compartment 118.

At step 414, the blockchain network 106 records information related to the quantity of medication present in the dispenser 102 after each dispensing period and information related to the requests from the first user to dispense the quantity of medication in the distributed ledger/blockchain protocol 114. At step 416, the blockchain network 106 updates the tokens assigned to the first user based on the dispensed quantity of medication. At step 418, the blockchain network 106 enables the first user to trade the tokens corresponding to unused quantity of medication for a monetary value with the guidance of the second user. In one embodiment, the medication comprises opioid drugs and the quantity of medication is a measure of morphine-equivalent units of the opioid drugs. Further, the blockchain network 106 is configured to create and store a usage history for each patient at the distributed ledger/blockchain protocol 114, and send alert regarding the usage of medication to at least one of the first user, the second user, the third user and a caretaker. The blockchain network 106 is configured to issue alerts or reports in the event of deviations in dispenser weight from the expected value. The deviation of dispenser weight could be determined using the weight sensor and usage history of medication. The blockchain network 106 is further configured to generate alerts or reports for unauthorized access or tampering. The blockchain network 106 is further configured to store and update patient data for each interaction. Each interaction contributes to the enhancement of the system's intelligence. The artificial intelligence module is configured to analyze and process patient data, including patient history, prior usage, socioeconomic data, case example data, and police data. The artificial intelligence module utilizes standards of usage and addiction to guide subsequent prescription decisions.

In certain embodiments, the weighing unit is factory-calibrated and field-calibratable to ensure traceable auditability of dispensed mass. A calibration routine may employ at least two traceable reference weights to estimate and persist linearization and bias parameters (k, b) for the load cell, optionally with temperature compensation using a stored lookup table. The control unit maintains a calibration certificate including: device identifier, firmware version, date/time of calibration, reference weights used, computed parameters, and residual error; the certificate is hashed and the resulting digest is committed to the ledger to bind subsequent telemetry to a known metrological state. During operation, the dispenser computes a rolling verification using an internal check mass or tare event; if drift exceeds a tolerance F (e.g., ±0.05 g or a policy-defined ppm), the system enters a degraded mode that inhibits dispensing or requires elevated authorization. Timekeeping for audit trails combines a secure monotonic counter resident in the secure element with a wall-clock synchronized via authenticated NTP and, where available, IEEE-1588 Precision Time Protocol (PTP). The control unit records both monotonic and wall-clock timestamps in each telemetry packet; clock discipline logic bounds drift by rate-of-change constraints and rejects backward jumps. When network synchronization is unavailable, the device continues using the monotonic counter and marks packets as “unsynchronized”; upon re-acquisition, the gateway or oracle reconciles wall-clock by bounded interpolation and commits an adjustment record to the ledger. In some embodiments, the dispenser also anchors time to the distributed ledger by referencing a recent block height and block timestamp within the authorization event, providing an external, tamper-evident time reference that improves auditability in intermittently connected environments.

In certain embodiments, the weighing unit comprises a strain-gauge load cell coupled to a high-resolution delta-sigma analog-to-digital converter having at least 20 effective bits over the expected dynamic range, yielding a nominal resolution of approximately 0.01 g at the packaged medication's operating mass. The control unit maintains calibration parameters including gain and offset and applies temperature compensation using a stored coefficient or table derived during factory calibration. During steady state, the weighing unit samples at a baseline cadence of at least 10 Hz and computes a rolling median to reject transient motion artifacts. Upon entry into an authorization sequence, the sampling cadence is increased to at least 50 Hz to capture fast mass transitions associated with removal of a unit dose. A low-latency finite-impulse response or single-pole IIR filter is applied to the oversampled stream, and the filtered signal is decimated to produce a measurement trace that is stored with pre- and post-event baselines. Quantization noise, ADC saturation, and outlier readings beyond a policy-defined sigma bound are flagged and excluded from dose calculations while remaining available in the diagnostic trace.

In certain embodiments, dispense detection is performed by comparing a continuously computed mass delta against a target dose value under a tolerance F established during calibration. The control unit records a pre-dispense baseline from a fixed window immediately preceding an authorization event and a post-dispense baseline from a window following closure of the enclosure or lapse of the dispense window, whichever occurs first. A dose is considered achieved when the absolute mass change crosses the target threshold within F and the derivative of the mass signal returns to within a quiescent band for a minimum settling time, thereby suppressing false positives due to vibration or user handling. If the mass delta fails to reach the target within a maximum window length Tₘₐₓ, or exhibits oscillation beyond a variance bound, the attempt is marked incomplete, the lock actuator is returned to the locked state, and the authorization is invalidated pending renewed approval. The computed pre- and post-baselines, the raw and filtered traces, the final mass delta, and the tolerance applied are included in the device-signed telemetry packet for audit.

In certain embodiments, tamper detection is effected by redundant channels that include an enclosure state sensor and inertial sensing. An enclosure sensor (e.g., reed or Hall switch) generates an event if an opening is detected while the dispenser is in the locked state or outside an active dispense window. A tri-axial accelerometer sampled at 100-200 Hz sets a tamper bit when resultant acceleration magnitude exceeds a threshold (for example, 0.5 g) for a persistence interval (for example, greater than 200 ms) or when the integrated tilt exceeds a policy limit indicative of inversion. Unexpected mass changes while locked that exceed a micro-delta threshold are classified as potential siphoning and also set the tamper bit. Tamper status is sticky until cleared by an authenticated procedure and is incorporated into the telemetry packet together with peak acceleration, duration, and enclosure state at the time of the event. Detection of a tamper event may inhibit further authorization events, require threshold (multi-party) authorization, or trigger an emergency audit mode as defined by policy.

In certain embodiments, the control unit implements a deterministic state machine that governs access and reporting. In a LOCKED state, the dispenser denies physical access and only accepts requests to verify authorization events. Receipt of a valid, unexpired authorization event whose cryptographic commitment matches the locally measured pre-dispense inventory causes a transition to AUTH_PENDING, during which the device performs final checks including calibration freshness, clock status, and tamper bit. Upon successful checks, the device transitions to DISPENSE, energizes the lock actuator to permit access, increases the weighing unit sampling cadence, and monitors the mass signal until the target dose is achieved within F or Tₘₐₓ elapses. Completion triggers transition to AUDIT, wherein the device compiles and signs a telemetry packet containing nonce, timestamps, pre- and post-baselines, mass delta, tamper status, firmware and calibration identifiers, and optional diagnostics, and submits the packet via the authenticated oracle. A validation error, tamper event, stale authorization, or calibration drift beyond tolerance diverts the flow to a DEGRADED state that restricts dispensing and requires elevated authorization or recalibration. Power-loss or communication faults are handled by idempotent re-entry semantics, with replay protection enforced by nonces and monotonic counters housed in the secure element, ensuring that partially completed transitions do not result in duplicate dispenses.

To enhance auditability and resilience, the device persists a circular buffer of recent high-rate measurement traces and state transitions in non-volatile memory, each entry indexed by a monotonically increasing counter and cross-referenced to the most recent authorization event identifier. A watchdog timer supervises the state machine and forces a safe return to LOCKED upon software fault, with a corresponding fault record included in the next telemetry packet. Time stamps recorded in each state transition include both a secure monotonic tick count and a wall-clock value disciplined by authenticated NTP or PTP when available; backward jumps and excessive drift are rejected by rate-limit logic and cause a transition to DEGRADED until time discipline is re-established. The foregoing device-side details provide concrete, measurable behavior that ties on-chain authorization to verifiable physical outcomes, thereby improving the integrity and auditability of the dispensing process relative to conventional server-polled or unauthenticated dispensers.

In certain embodiments, the communications between the dispenser, gateway, oracle, and backend services employ TLS 1.3 with mutual authentication using X.509 device and service certificates. Handshakes use ephemeral ECDHE suites to provide forward secrecy; certificate pinning and OCSP stapling mitigate interception and downgrade attempts. Device identity and integrity are proven by remote attestation: the control unit, via its secure element, responds to a nonce-based challenge with signatures over measured boot artifacts (e.g., firmware hash, configuration digest, secure element serial), which the attestation service validates before accepting telemetry or issuing authorization events. Firmware updates are delivered as signed artifacts bearing a version number and release manifest; the bootloader enforces signature verification, anti-rollback monotonic counters, and atomic swap with fallback on failure. Backend signing, decryption, and key-wrapping operations are performed within a hardware security module meeting FIPS 140-3 (e.g., Level 3) with dual-control and role separation; keys are rotated under policy, and compromised credentials are revoked via a published certificate revocation list and on-chain governance transaction. All security-relevant events—including failed attestation, certificate expiry, firmware update success/failure, and TLS handshake anomalies—are logged and, in some embodiments, summarized as Merkle-authenticated digests committed to the ledger to provide a tamper-evident security audit trail.

In preferred embodiments, artifacts recorded to the distributed ledger are pseudonymous digests that omit protected health information (PHI). Subject identity and other regulated data are maintained in a segregated data store under role-based access control and attestation, with access events logged and periodically audited. On-chain entries reference the segregated records only via cryptographic commitments (e.g., salted digests or Merkle proofs), thereby enabling verification of integrity and ordering without disclosing PHI.

In certain embodiments, the tokenized dose entitlements are managed to reflect clinical policy without implying an open-market financial exchange. By default, entitlements are non-transferable except among a restricted policy graph of approved principals (e.g., prescriber, dispensing facility, administrator) and are associated with a specific patient wallet. Upon verified dispense, the smart contract decrements the active entitlement. Unused entitlements may be reconciled by one or more of: (i) burn, wherein the smart contract extinguishes remaining units upon expiry, revocation, or verified return; (ii) reissue, wherein remaining units are reallocated by the prescriber to a replacement prescription or alternative package under policy; and (iii) stable redemption, wherein remaining units are redeemed into a non-monetary compliance credit or care-coordination benefit that is recorded on-chain but is not convertible to currency or tradeable on secondary markets. In certain embodiments, the contract enforces non-transferability by rejecting transfers to addresses outside the approved policy graph and by binding entitlements to a patient wallet keyset. The foregoing reconciliation mechanisms emphasize clinical control, auditability, and safety while reducing characterization of the system as a financial exchange.

Advantageously, the system tracks a usage history of each patient, and send alert regarding the usage of medication to prevent misuse of the medication. Further, incentivizing medication management encourages the patient to rely less on opioid type medications. Further, the system of the present invention provides an analysis on the availability of opioid in a location based on the tokens assigned and transferred between individuals.

In one embodiment, the present invention provides for non-transitory computer-readable medium storing instructions that, when executed by a node of the blockchain network, cause the node to: validate dispenser-signed telemetry; enforce dosing limits; emit authorization events; and update tokenized entitlements with Merkle-authenticated commitments to dispenser state.

Although the features, functions, components, and parts have been described herein in accordance with the teachings of the present disclosure, the scope of coverage of this patent is not limited thereto. On the contrary, this patent covers all embodiments of the teachings of the disclosure that fairly fall within the scope of permissible equivalents.

Many modifications and other implementations of the disclosure set forth herein will be apparent having the benefit of the teachings presented in the foregoing descriptions and the associated drawings. Therefore, it is to be understood that the disclosure is not to be limited to the specific implementations disclosed and that modifications and other implementations are intended to be included within the scope of the appended claims. Although specific terms are employed herein, they are used in a generic and descriptive sense only and not for purposes of limitation.

What is claimed is:

1. A blockchain based system for medication management and incentivizing medication management, comprising:
a medication dispenser comprising at least one compartment, a control unit, and a dispensing unit, wherein the compartment is configured to store a quantity of medication, the medication dispenser is associated with a first user;
a blockchain network in communication with the dispenser;
a first user device in communication with the blockchain network and the dispenser, the first user device is associated with the first user, and
a second user device in communication with the blockchain network, the second user device is associated with a second user, the blockchain network utilizes smart contracts or similar mechanisms to:
define policy parameters comprising a predefined limit and a current period within a policy graph;
enable the second user to prescribe a quantity of medication to the first user and assign tokenized dose entitlements corresponding to the prescribed quantity to the first user, the entitlements being non-transferable outside the policy graph and not convertible to currency;
receive, from the first user via the first user device, a request to dispense a quantity of medication;
retrieve information corresponding to (i) the requested quantity of medication to be dispensed and (ii) a quantity of medication present in the compartment;
determine whether the requested quantity of medication is within the predefined limit for the current period;
approve the request to dispense the requested quantity of medication in response to determining that the requested quantity is within the predefined limit for the current period;
record, in a distributed ledger protocol of the blockchain network, an authenticated digest of information related to the quantity of medication present and dispensed;
update the tokenized dose entitlements assigned to the first user based on the dispensed quantity of medication; and
enable the first user to reconcile unused entitlements via on-chain burn or prescriber-directed reissue or other non-monetary reconciliation confined to the policy graph.

2. The system of claim 1, wherein the medication comprises opioid drugs and the quantity of medication is a measure of morphine-equivalent units of the opioid drugs.

3. The system of claim 1, wherein the medication dispenser further comprises a locking unit configured to lock the dispensing unit to prevent dispense of medications without authorization.

4. The system of claim 1, wherein the control unit is configured to enable the locking unit to unlock the dispensing unit based on the approval of the request received from the blockchain network.

5. The system of claim 1, wherein the control unit is configured to enable the dispensing unit to dispense the requested quantity of medication.

6. The system of claim 1, wherein the system further comprises a load cell/weight sensor configured to output a mass measurement related to the quantity of medication in the compartment.

7. The system of claim 1, wherein the first user includes a patient and the second user includes a healthcare provider.

8. The system of claim 1, wherein the medication dispenser is linked to the first user device via an authentication system, and wherein the authentication system includes at least one of a biometric scan-based authentication system, a two-factor/multi-factor authentication system, an electronic authentication and a third party authentication.

9. The system of claim 1, further comprises a third user device in communication with the blockchain network, the third user device is associated with a third user, the blockchain network is configured to enable the third user to allocate and transfer a predefined number of tokens for a predefined quantity of medication to the second user, wherein the third user is an administrator.

10. The system of claim 1, wherein the blockchain network is configured to create and store a usage history for each patient at the blockchain, and send alert regarding the usage of medication to at least one of the first user, the second user, the third user and a caretaker, and issue at least one of alerts or reports in the event of deviation in dispenser weight from an expected value, and on unauthorized access or tampering of the dispenser, wherein the blockchain network is configured to store and update patient data for each interaction, wherein subsequent interactions contribute to the enhancement of the system's intelligence.

11. The system of claim 1, further comprises an artificial intelligence module configured to analyze and process patient data, including patient history, prescriber history, prior usage, socioeconomic data, case example data, and police data, wherein the artificial intelligence module utilizes standards of usage and addiction to guide subsequent prescription decisions.

12. A blockchain based method for medication management and incentivizing medication management, comprising the steps of:
providing a system comprising a medication dispenser and a first user device associated with a first user, a blockchain network in communication with the medication dispenser and the first user device, and a second user device in communication with the blockchain network, wherein the medication dispenser comprising at least one compartment, a control unit, a weighing unit, a locking unit and a dispensing unit, wherein the compartment is configured to store a quantity of medication, wherein the second user device is associated with a second user, wherein the blockchain network utilizes smart contracts or other types of automated, self-regulating contracts;
enabling, at the blockchain network, the second user to prescribe a quantity of medication to be filled in the compartment and transfer a number of tokens corresponding to the quantity of medication prescribed to the first user, via the second user device, wherein each token comprises a monetary value;
receiving, at the blockchain network, a request from the first user, via the first user device, to dispense a quantity of medication;
retrieving, at the blockchain network, at least one of an information corresponding to a quantity of medication stored after a previous dispense period and information corresponding to the quantity of medication present in the compartment;
retrieving, at the blockchain network, the information related to the requested quantity of medication for a current period of time;
determining, at the blockchain network, if the requested quantity of medication is within a predefined limit for the current period of time;
approving, at the blockchain network, the request to dispense the requested quantity of medication when the requested quantity of medication is within the predefined limit for the current period of time;
record, at the blockchain network, information related to the quantity of medication present in the medication dispenser after each dispensing period and information related to the request from the first user to dispense the quantity of medication in a distributed ledger/blockchain protocol;
updating, at the blockchain network, the tokens assigned to the first user based on the dispensed quantity of medication, and
enabling, at the blockchain network, the first user to trade the tokens corresponding to an unused quantity of medication for a monetary value with a guidance of the second user.

13. The method of claim 12, wherein the medication comprises opioid drugs and the quantity of medication is a measure of morphine-equivalent units of the opioid drugs.

14. The method of claim 12, wherein the medication dispenser is linked to the first user device via an authentication system, and wherein the authentication system includes at least one of a biometric scan-based authentication system, a two-factor or multi-factor authentication system, an electronic authentication and a third party authentication.

15. The method of claim 12, further comprising the step of: locking the dispensing unit, via the locking unit, to prevent dispense of medications without authorization.

16. The method of claim 15, further comprising the step of: enabling the locking unit, via the control unit, to unlock the dispensing unit based on the approval of the request received from the blockchain network.

17. The method of claim 15, further comprising the step of: enabling the dispensing unit, via the control unit, to dispense the requested quantity of medication, and transmitting information related to the quantity of medication in the compartment via the weighing unit.

18. The method of claim 12, wherein the system further comprises a third user device in communication with the blockchain network, wherein the third user device is associated with a third user, wherein the third user is an administrator.

19. The method of claim 12, further comprising the steps of:
enabling, at the blockchain network, the third user to allocate and transfer a predefined number of tokens for a predefined quantity of medication to the second user;
creating and storing, at the blockchain network, a usage history for each patient at the distributed ledger/blockchain protocol;
sending, via the blockchain network, alert regarding the usage of medication to at least one of the first user, the second user, the third user and a caretaker;
storing and updating, via the blockchain network, patient data for each interaction, wherein subsequent interactions contribute to the enhancement of the system's intelligence;
analyzing and process, via an artificial intelligence module, patient data, including patient history, prior usage, socioeconomic data, case example data, and police data, wherein the artificial intelligence module utilizes standards of usage and addiction to guide subsequent prescription decisions, and
issuing, via the blockchain network, at least one of alerts or reports in the event of deviation in dispenser weight from an expected value, and on unauthorized access or tampering of the dispenser.

20. A computer-implemented method of controlling a medication dispenser using a distributed ledger, comprising: maintaining, by a smart contract, tokenized dose entitlements; verifying, responsive to a dispense request, policy constraints and threshold authorizations; emitting an authorization event that commits to a pre-dispense dispenser state; authenticating, by a control unit, the authorization event and verifying the committed state against locally measured sensor data; unlocking a lock actuator only upon successful verification;
measuring a mass change during a dispense window; generating and device-signing a telemetry packet with nonce and timestamps; submitting the telemetry via an authenticated oracle; and committing, by the smart contract, a Merkle-authenticated digest of accepted telemetry while updating the entitlements and rejecting stale or replayed telemetry.
